(19) United States
(12) Patent Application Publication
(10) Pub. No.: US 2022/0039358 A1
(43) Pub. Date: Feb. 10, 2022

(54) System and Method for Determining Caloric Requirements of an Animal Based on a Plurality of Durational Parameters

(71) Applicant: Hill's Pet Nutrition, Inc., Topeka, KS (US)

(72) Inventors: Susan Wernimont, Lawrence, KS (US); Kristen Carnagey, Lawrence, KS (US); Jodi Vondran, Wamego, KS (US); Mark Szczerbaniewicz, Oakland, NJ (US); Robin Thompson, Newcastle upon Tyne (GB)

(73) Assignee: Hill's Pet Nutrition, Inc., Topeka, KS (US)

(21) Appl. No.: 17/383,750

(22) Filed: Jul. 23, 2021

Related U.S. Application Data
(60) Provisional application No. 63/060,793, filed on Aug. 4, 2020.

(51) Int. Cl. A01K 29/00; A01K 5/02; A01K 11/00; G05B 19/4155

(52) U.S. Cl. CPC A01K 29/005; A01K 5/0275; G05B 2219/45113; G05B 19/4155; A01K 11/008

(57) ABSTRACT

A system, apparatus, and/or method of determining a caloric requirement of an animal may be provided. In an aspect, animal characteristic data comprising a breed of an animal or a condition of the animal may be received. Durational parameters of the animal for a first predetermined time duration may be received. The durational parameters may include at least one of an activity level of the animal, a weight of the animal, or an amount of calories consumed by the animal during the first predetermined time period. A caloric requirement of the animal during a second predetermined time period may be determined. The caloric requirement of the animal may be based on the animal characteristic data of the animal and/or the durational parameters of the animal during the first predetermined time period. The caloric requirement of the animal during the second predetermined time period may be displayed via a display device.

### CROSS-REFERENCE TO RELATED APPLICATIONS

The present application claims priority to U.S. Provisional Patent Application Ser. No. 63/060,793, filed Aug. 4, 2020, the entirety of which is incorporated herein by reference.

### BACKGROUND

Veterinarians and animal caregivers have a need to know the appropriate amount of calories, via food, provided to a pet. Obesity in pets (such as dogs and cats) is known, likely due to pet owners providing an amount of food inappropriate to their pet's size, life stage, and activity level. Currently, caloric requirements for pets are calculated based on a daily caloric requirement that is typically based on published (i.e., static) equations. The caloric requirements of a pet can be based on one or more conditions. Determining the correct daily caloric requirement typically requires guesses to be made about the conditions associated with the pet, which is often not observed or calculatable by pet owners. Ultimately, because of the inability to accurately determine the amount of calories needed by a pet, most veterinarians and animal caregivers ultimately feed an amount of food based on a “best guess” and monitor the animal's weight to determine if the guess was correct. While practical, this solution allows for weeks or months of excessive or inadequate feeding before a problem is detected. Further, such solution relies on frequently, and manually, weighing of the pet to measure success.

What is desired is a tool that provides (1) an objective and accurate assessment of conditions related to a pet, and (2) provides a near-real time output of the conditions that would greatly enhance efforts to appropriately manage pet weight. Such a tool may be a system, apparatus, and/or method that uses measurements of conditions related to a pet to provide energy (and thus food) requirements that are based on an objective assessment of such conditions.

### BRIEF SUMMARY

A system, apparatus, and/or method of determining a caloric requirement of an animal, such as a pet, may be provided. In an aspect, animal characteristic data comprising a body type (e.g., body shape, such as long, short, thin, stocky) of an animal, a breed of an animal, and/or a condition of the animal may be received via one or more processors. Durational parameters of the animal for a first predetermined time duration may be received via one or more elements. The durational parameters may include at least one of an activity level of the animal during the first predetermined time period, a weight of the animal during the first predetermined time period, or an amount of calories consumed by the animal during the first predetermined time period. A caloric requirement of the animal during a second predetermined time period may be determined. The caloric requirement of the animal may be based on the animal characteristic data of the animal and/or the durational parameters of the animal during the first predetermined time period. The caloric requirement of the animal during the second predetermined time period may be caused to be displayed via a display device.

In an aspect, one or more elements may be configured to receive durational parameters of the animal for a first predetermined time duration. The durational parameters may include at least one of an activity level of the animal during the first predetermined time period, a weight of the animal during the first predetermined time period, or an amount of calories consumed by the animal during the first predetermined time period. One or more processors may be configured to: receive animal characteristic data comprising a body type of the animal, a breed of the animal, and/or a condition of the animal; determine a caloric requirement of the animal during a second predetermined time period, wherein the caloric requirement of the animal being based on the animal characteristic data of the animal and the durational parameters of the animal during the first predetermined time period; and cause the caloric requirement of the animal during the second predetermined time period to be displayed via a display device.

### BRIEF DESCRIPTION OF THE DRAWINGS

The present invention will become more fully understood from the detailed description and the accompanying drawings, wherein:

FIG. 1 is a block diagram of a system having a plurality of modules configured to collect and analyze the behavior of an animal, as described herein;

FIG. 2 is a perspective view of an example activity collar, as described herein;

FIGS. 3A and 3B are depictions of animals wearing an example activity collar; as described herein;

FIG. 4 is a perspective view of an example feeding dish, as described herein;

FIG. 5 is a perspective view of an example weighing scale, as described herein;

FIGS. 6A-6B are example screenshots of a use of the system of FIG. 1; and

FIG. 7 is an example process of determining a caloric requirement of an animal, as described herein.

### DETAILED DESCRIPTION

The following description of the preferred embodiment(s) is merely exemplary in nature and is in no way intended to limit the invention, its application, or uses.

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top,” and “bottom” as well as derivatives thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments illustrating some possible non-limiting combination of features that may exist alone or in other combinations of features; the scope of the invention being defined by the claims appended hereto.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

The system, method, and apparatus provide an automatic and accurate amount of calories (e.g., via food sources, such as pet foods) required and/or recommended of an animal, such as a pet (e.g., dog, cat, etc.), farm animal (e.g., horse, cow), bird, fish, and the like. The amount of calories required may be based on many factors relating to the animal, such as the activity level of the animal (e.g., as assessed by a wearable sensor), the weight of the animal, the amount of calories consumed by the animal, the movements of the animal, the breed of the animal, the size and/or body type (e.g., long, tall, thin, stocky) of the animal, etc. As described herein, one or more of the factors relating to the animal (e.g., the activity level of the animal, the weight of the animal, and/or the amount of calories consumed by the animal) may be received and/or determined automatically.

Veterinarians and animal caregivers (i.e., pet owners or pet parents) may need to know the appropriate amount of calories to provide an animal, in which the calories may be provided via food consumed by the animal. For example, by providing (e.g., automatically providing) the appropriate amount of food to an animal, the health of the animal may be positively impacted, such as by preventing obesity in the animal or providing nutrition therapy (e.g., for obesity) of the animal. The prevalence of obesity in pets (e.g., dogs and cats) has been identified as a health concern. Such prevalence of obesity in pets suggests that many pet owners are providing the pet an amount of food inappropriate for the pet's size, life stage, activity level, and the like. By providing the appropriate amount of food (e.g., calories) to an animal, the prevalence of health disorders (e.g., disorders caused by obesity) may be decreased or prevented.

Caloric requirements for pets may be determined in one or more ways and/or via one or more parameters. For example, as described herein, the caloric requirements for pets may be determined based on the activity level of the animal, the weight of the animal, the amount of calories consumed by the animal, the movements of the animal, the breed of the animal, the body type of the animal, the size of the animal, etc. Caloric requirements for pets may be based on a Daily Energy Requirement (DER) value. The DER value may be calculated as a Resting Energy Requirement (RER) value multiplied by one or more other parameter values. For example, the RER value may be multiplied by a factor F, as shown in Equation 1 below.

DER=RER×F. Equation 1:

The value of RER may include units of kcals/day. The value of RER may be based on the metabolic body weight of a pet. In examples, metabolic body weight may be weight (e.g., in kilograms). In examples RER may be expressed as KG0.75. An average RER for dogs and cats may be 70 kcals/day per KG0.75.

Factor F, which may be used to obtain DER, may incorporate one or more factors related to the animal, such as the animal's activity level, neuter status, life stage, health status, and the like. Factor F may be obtained from one or more lists (e.g., lists containing predetermined values). For example, for neutered adult dogs the factor may be 1.6, for intact adult dogs the factor may be 1.8, for inactive/obese prone dogs the factor may be 1.2-1.4, for critical care dogs the factor may be 1.0, and for heavy work dogs the factor may be 5.0-11.0. In cats, the factor for neutered adult cats may be 1.2-1.4, for intact adult cats the factor may be 1.4-1.6, for inactive/obese prone cats the factor may be 1.0, and for critical care cats the factor may be 1.0. The value of the factor may also, or alternatively, vary according to other conditions of the animal, such as a condition (e.g., pregnancy condition) of the animal, whether the animal is lactating, and the like.

Pet activity level may contribute to the choice of a factor for the calculation of DER. Pet activity may be difficult for animal caregivers and veterinarians to determine (e.g., estimate). For example, estimating the correct factor to use may require guesses to be made about the animal's level of activity, which may not be observed by pet owners. Further, activity factors may not be published in ways that reflect several different conditions co-occurring. Thus, a veterinarian or animal caregiver may have to combine factors to represent (e.g., accurately represent) the condition of a pet. For example, a veterinarian or animal caregiver may have to combine factors such as a cat being an obese prone lactating female cat or a dog being a neutered young dog engaging in heavy work. Such combining of factors may require guesswork in conventional systems. Ultimately, because of the inability to accurately estimate the amount of food needed by a pet, veterinarians and animal caregivers may feed an amount of food based on a best guess and monitor the animal's weight to determine if the guess was correct. While guessing strategies may be practical, such solutions allow for weeks or months of excessive or inadequate feeding before a problem is detected and relies on frequently manual weighing of the pet to measure success. To compound the problem, pet owners and vets may fail to recognize obesity in animals when present. As a result, most pets may not be fed an appropriate amount of food, as evidenced by obesity rates.

Examples herein describe a system, method, and/or apparatus that overcome the limitations in which calorie requirements are assessed for pets. For example, examples herein describe a tool that provides (1) an objective, automatic, and accurate assessment of conditions relating to a pet and (2) a near-real time output of the conditions that enhances efforts to appropriately manage pet weight. The tool may use measurements of the animal's activity, weight, consumption habits, breed, body type, size, etc., to provide energy (and thus food) requirements that may be based on an objective assessment of activity. The automatic and accurate determination of caloric requirements of an animal may result in many benefits to the animal, especially if the veterinarian and/or animal's caretaker take corrective action as result of the determined caloric requirements. For example, the weight of the animal may be properly managed, leading to less health issues as a result of obesity in the animal.

The system may include one or more devices and/or mechanisms for receiving, determining, storing, and/or transmitting information relating to the animal. For example, the system may include one more devices worn by the animal and/or coupled to an article worn by the animal, such as a collar, harness, bracelet, anklet, belt, earring, headband, coat, boot, decorative clothing (e.g., ribbon), sweater, hat, etc. In other examples the devices and/or mechanisms may be associated with an animal and not worn by the animal. Such devices may include weight scales (e.g., smart scales for weighing pets), pet feeders, waste areas (e.g., litter boxes), implantable devices, and the like. For example, one or more of the devices may be implanted underneath the skin, such as a subdermal implant. As understood by those of skill in the art, smart scales may include scales that are enabled with Wi-Fi, Bluetooth (e.g., Bluetooth classic or Bluetooth Low-Energy), cellular, and/or other data transmission modalities that may enable the scale to communicate with one or more devices associated with the pet (such as an accelerometer) and/or that may be used to recognize the pet.

A device (e.g., a recognition device) may identify the animal within the system. The animal may be linked to an animal profile. Activities (e.g., movements, feedings, etc.) and/or parameters (e.g., weight) associated with the animal's profile may be monitored, tracked, and/or electronically recorded (e.g., automatically monitored, tracked, and/or electronically recorded) on a predefined frequency (e.g., on a daily, weekly, monthly, yearly basis). The animal's activities and/or other parameters may be used to determine the animal's activity level, movements, weight, feeding habits, etc., as described herein. The animal's activities and/or other parameters may be monitored, tracked, and/or recorded without disturbing the animal or disrupting its natural behavior.

The monitoring of the animal's activities and/or other parameters may be performed via collection of one or more types of data. The data may include motion data of the animal (e.g., detected via an accelerometer), location data of the animal (e.g., detected via a global positioning system (GPS) device, a proximity beacon, cellular triangulation techniques, etc.), weight data of the animal, feeding data of the animal, and the like. The data may be collected and/or monitored during one or more pet activities, such as while the pet is walking, running, jogging, crawling, trotting, cantering, galloping, ambling, resting, eating, drinking, standing, and the like. Movement data may relate to the animal moving in one or more directions, such as in a forward direction, reverse direction, sideward direction, up/down direction, etc. Movement data may relate to a gait of the animal, the speed at which the animal is moving, the duration in which the animal is moving, etc. Collected data may be stored in a repository that may be accessible to animal caregivers, veterinarians, and the like. The data may be accessible via a portable electronic device (e.g., an application of a portable electronic device) and/or a server.

A portable electronic device may be one or more of a number of devices, including without limitation, a smart phone, a cell phone, a tablet computer, a personal digital assistant (“PDA”), a laptop computer, router, edge computing device, etc. The data (e.g., activity level, movement data, location data, etc.) of the animal may be analyzed to determine caloric (e.g., calorie) requirements of the animal. The determined caloric requirements may be provided to veterinarians and/or pet owners based on the data (e.g., activity level, movement data, location data, etc.). The activity data and/or other parameter data (e.g., weight data, feeding data) of the pet may be collected and/or generated over time, for example, for statistical processing of the animal's caloric requirements. The data may be compared with previously collected and/or stored data for purposes of correlating the animal's activity levels, movements, weights, and/or feedings with the caloric requirements of the animal. The previously collected and/or stored data may relate to the animal that is being monitored and/or the previously collected and/or stored data may relate to another animal (e.g., a similarly situation animal, such as an animal of the same body type, breed, weight, condition, etc.) for comparison purposes.

An animal's caloric requirements, such as whether the animal is consuming too little, just enough, or too many calories, may be determined based on the activity levels, movements, weights, and/or feedings of the animal. The animal's caloric requirements may be recorded. To determine the animal's calorie requirements, parameters indicative of the animal's activity levels, movements, weights, and/or feedings may be monitored and/or recorded. Such parameters may include the speed in which the animal moves, acceleration of the animal at one or more times, duration in which the animal moves within a predetermined time, distance traveled by the animal, direction in which the animal moves, etc.

Alternatively, or additionally, such parameters may include the weight of the animal, the height of the animal, the length of the animal, the amount consumed by the animal, times of day in which the animal eats, etc. As an example, an animal running multiple hours per day and/or weighing above a predefined number of pounds may have a higher caloric requirement than an animal that runs less than one hour per day and/or that weighs below a predefined number of pounds. As described herein, the caloric requirement may be based on attributes other than the activity level of the animal and/or the weight of the animal, such as the body type (e.g., body shape, such as long, short, thin, stocky) of the animal, breed of animal, gender of animal, pregnancy condition of the animal, body weight index (BFI), leg lengths, head size (e.g., circumference of the head), etc. For example, a pregnant animal and/or a large breed animal that runs less than an hour a day may have a higher caloric requirement than a non-pregnant animal and/or a small breed animal (e.g., non-similarly situated animal) that runs more than an hour a day. As described herein, characteristics of the animal including the breed of the animal and/or the condition of the animal may be referred to as animal characteristics of the animal. Parameters relating to the activity level of the animal during a predetermined duration (e.g., time period, such as a day), a weight of the animal during a time period, an amount of calories consumed by the animal during the time period, and/or the like may be referred to as durational parameters of the animal.

The application of statistical methods may be used to derive information about the animal's caloric requirements based on the activity level, weight, calories consumed, condition, and/or movement of the animal. For example, an animal running a minimum and/or maximum amount of time during a time period, consuming a minimum and/or maximum amount of calories during a time period, and/or weighing a minimum and/or maximum number of pounds during a time period (e.g., per day, per week, per month, etc.) may be expected to have a predetermined caloric requirement during a future time period that may be the same or different than the time period in which the animal is monitored and/or for which the caloric requirement is determined. If parameters relating to the animal deviate from a defined parameter (e.g., the animal runs for less than, or more than, an amount defined for the animal), it may be determined that the animal may require less calories or more calories than the caloric requirement previously predetermined for the animal.

As described herein, characteristics of the animal may be used to determine caloric requirements of the animal. Such characteristics may include the species, breed, age, gender, geographic location, size/weight, etc., of the animal. For example, an animal of the dog species may be expected to move faster and/or farther than an animal of the cat species. As a result, a running distance per day that substantially increases the caloric requirements for a cat may not similarly substantially increase the caloric requirements for a dog running a similar distance per day. In another example, a first dog having a weight that is greater than a second dog may have a higher caloric requirement than the second dog, at least until the weight of the first dog is reduced and/or brought to a desired level.

Parameters determined, identified, received, and/or transmitted may be recorded. The parameters may be recorded continuously, for example, from the moment of system activation throughout the animal's life. In other examples, the parameters may be recorded for a predefined time period (e.g., for a day, a week, a month, etc.), on a predefined frequency (e.g., every weekday), etc.

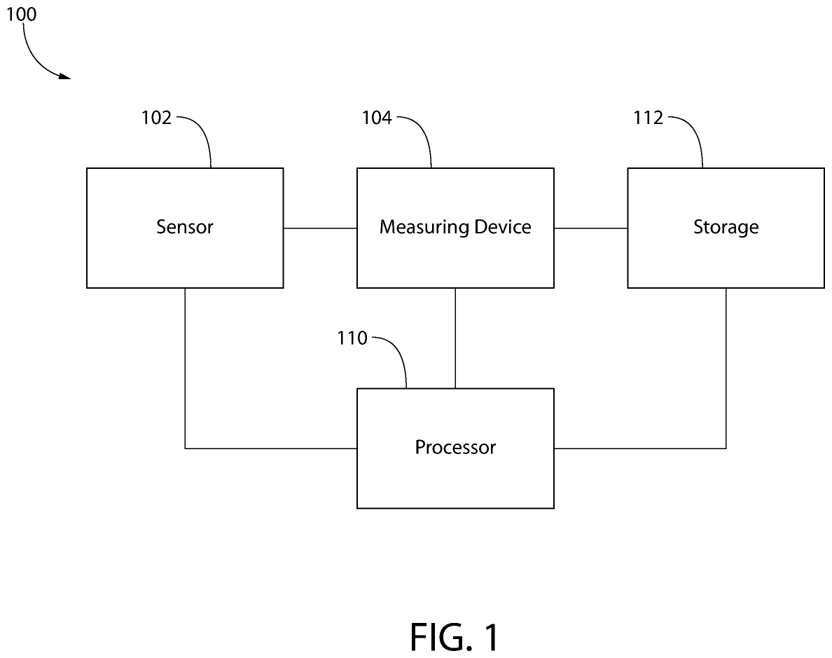

FIG. 1 shows an example system for determining caloric requirements for an animal based on the activity levels, movements, weights, and/or feedings of the animal. System 100 may include a sensor 102, a measuring device 104, and/or a storage device 112.

Sensor 102 may be configured to detect a location of the animal, to detect the motion (or stillness) of the animal, to detect an orientation of the animal, etc. Sensor 102 may be one or more of a variety of form factors, including, but not limited to, an accelerometer, a gyroscope, a magnetometer, weighing scales, weight transducers, force transducers, displacement transducers, pressure transducers, weight sensors, force sensors, displacement sensors, pressure sensors, load cells, photographic cameras, video cameras, camcorders, contact thermometers, non-contact thermometers, and a combination thereof. In addition, or alternatively, sensor 102 may be one or more of optical sensors, optical reflecting sensors, LED/photodiode pair optical sensors, LED/phototransistor pair optical sensors, laser diode/photodiode pair optical sensors, laser diode/phototransistor pair optical sensors, optocouplers, optical fiber coupled optical sensors, magnetic sensors, weight sensors, force sensors, displacement sensors, pressure sensors, various proximity sensors, such as inductive proximity sensors, magnetic proximity sensors, capacitive proximity sensors, and/or a combination thereof. Sensor 102 may include communication circuitry, such as Bluetooth, RFID, Wi-Fi and other wireless technologies. Sensor 102 may communicate with one or more devices, for example, sensor 102 may communicate with a server.

Measuring device 104 may be configured to measure a characteristic related to the animal. Measuring device 104 may be implemented in one or more of a variety of form factors, including, but not limited to, weighing scales, weight transducers, force transducers, displacement transducers, pressure transducers, weight sensors, force sensors, displacement sensors, pressure sensors, real time clocks, timers, counters, and/or a combination thereof. Measuring device 104 may include communication circuitry, such as Bluetooth, RFID, Wi-Fi and other wireless technologies. Measuring device 104 may communicate with one or more devices, for example, measuring device 104 may communicate with a server.

Storage device 112 may be configured to store data provided to and/or from system 100. The data may include motion data, activity level data, weight data, feeding data, and/or location data provided by the sensor 102, for example. Example storage devices 112 may be memory devices, data storage devices, and a combination thereof, such as memory chips, semiconductor memories, Integrated Circuits (IC's), non-volatile memories or storage device such as flash memories, Read Only Memories (ROM's), Erasable Read Only Memories (EROM's), Electrically Erasable Read Only Memories (EEROM's), Erasable Programmable Read Only Memories (EPROM's), Electrically Erasable Programmable Read Only Memories (EEPROM's), an Electrically Erasable Programmable Read Only Memory (EEPRO), volatile memories such as Random Access Memories (RAM's), Static Random Access Memories (SRAM's), Dynamic Random Access Memories (DRAM's), Single Data Rate memories (SDR's), Dual Data Rata memories (DDR's), Quad Data Rate memories (QDR's), microprocessor registers, microcontroller registers, CPU registers, controller registers, magnetic storage devices such as magnetic disks, magnetic hard disks, magnetic tapes, optical memory devices such as optical disks, compact disks (CD's), Digital Versatile Disks (DVD's), Blu-ray Disks, Magneto Optical Disks (MO Disks) and/or a combination thereof. In an example, the storage device comprises a semiconductor RAM IC for an intermediate recording of the behavior, health, and/or characteristics of the animal, and then transfer of the data to a flash memory IC for non-volatile recording. Storage device 112 may be an external memory device, such as a USB flash memory, an external hard drive, etc.

System 100 may include a processor 110 configured to receive data relating to the animal. For example, processor 110 may receive animal characteristic data of the animal including the breed of the animal, a condition of the animal, the size of the animal, age of the animal, the location of the animal, etc. Processor 110 may be configured to calculate and/or process data provided to system 100. For example, processor 110 may be configured to determine the calories required of an animal (e.g., based on an activity level of the animal), foods to be recommended to the animal (e.g., based on the amount of recommended calories), etc., as described herein. Example processors may be electronic circuits, systems, modules, subsystems, sub modules, devices and combinations thereof, such as Central Processing Units (CPU's), microprocessors, microcontrollers, processing units, control units, tangible media for recording and/or a combination thereof. Storage device 112 may be configured to store derived data from the processor 110. Processor 110 may include communication circuitry, such as Bluetooth, RFID, Wi-Fi, and other wireless technologies. Processor 110 may communicate with one or more devices, for example, processor 110 may communicate with a server.

In an example, measuring device 104, storage 112, and/or sensor 102 may be considered elements of the example system for determining caloric requirements for an animal. Sensor 102, measurement device 104, and/or storage 112 may be assembled in a number of configurations, including in a stand-alone apparatus. In another example, one or more of sensor 102, measurement device 104, storage 112, and processor 110 may each be assembled in a stand-alone apparatus. In other examples, one or more of the processor 110 and/or storage 112 may be configured as remote devices, such as remote servers (e.g., cloud storage devices). Although FIG. 1 shows a connection between processor 110 and each of sensor 102, measuring device 104, and storage 112, examples should not be so limited. In examples one or more of the devices may communicate with one or more (including any, or none) of the other devices. For example, sensor 102 may communicate with processor 110 and storage 112, sensor 102 may not communicate with storage 112, etc. One or more devices may be added and/or removed from system 100. For example, additional sensors 102 may be added to system 100 and/or storage 112 may be removed from system 100.

Data relating to the animal may be processed and/or recorded for a determination of the animal's caloric requirements. For example, the amount of times, durations, speed, directions, etc., in which an animal walks, jogs, runs, and/or rests may be used to determine an activity level of the animal and/or the caloric requirements of the animal, etc. A weight of an animal, feedings of the animal, a body temperature of an animal, a breed of an animal, an age of the animal, the date and/or time of an event (e.g., a walking, running of the animal) relating to an animal, etc. may be used to determine caloric requirements of an animal. One or more activities of the animal may be recorded via a video recording, picture, and/or audio recording and/or may be processed.

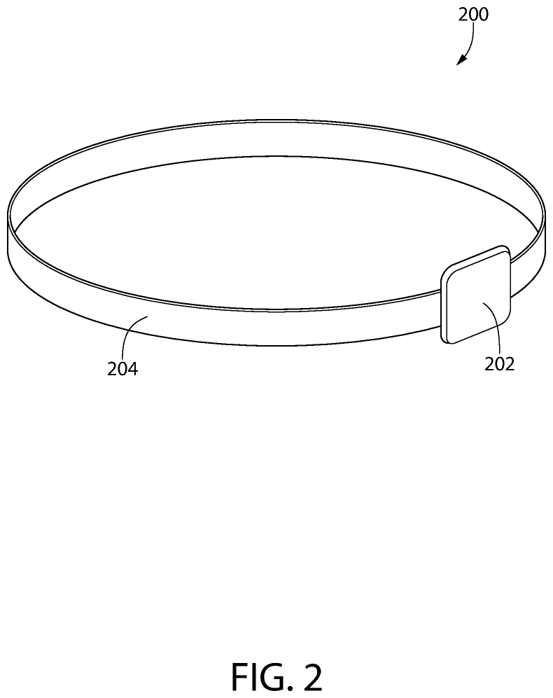

FIG. 2 is a perspective view of an example mechanisms 200 worn by an animal. Although FIG. 2 shows mechanism 200 having a collar 202, it should be understood that mechanism 200 may be one or more form factors worn by an animal, constraining the animal, weighing the animal, feeding the animal, implanted within the animal (e.g., subdermal implant), and/or proximate the animal. For example, mechanism 200 may be worn on one or more of the head of the animal, the ears of the animal, the neck of the animal, the torso of the animal, limbs (e.g., arms, legs) of the animal, the tail of the animal, and the like. As an example, mechanism 200 may be a collar, harness, bracelet, anklet, belt, earring, headband, weighing scale, feeding system, and the like. In other examples devices that may house or couple to an electronic device may include one or more attachment mechanisms, such as a coat, boot, decorative clothing (e.g., ribbon), sweater, hat, etc. Mechanism 200 may be used to constrain the animal, feed the animal (such as part of a water dish or a food bowl), weigh the animal, store information about the animal, process information about the animal, and/or transmit information relating to the animal.

Mechanism 200 may be linked to a particular animal (e.g., may be linked to a profile of a particular animal). Mechanism 200 may include circuitry 202 that may include a processor, storage, wireless communication hardware, one or more sensors (e.g., accelerometers, gyroscopes, magnetometers, etc.), GPS, temperature sensors, moisture detectors, biometric sensors, weight scales, feeding systems, etc. The wireless communication hardware may include a transmitter and a receiver. For example, the wireless communication hardware of the mechanism 200 may include a low energy communication device, such as Bluetooth Low Energy or RFID. The mechanism 200 may include a memory for storing data.

An accelerometer located on the mechanism 200 may be configured to measure one or more motions of the animal. For example, the accelerometer may measure accelerations of the animal, changes in velocity of the animal, and/or changes in position of the animal. A gyroscope may be configured to measure changes in orientation of the animal and/or changes in rotational velocity of the animal. A magnetometer may be configured to measure orientation (e.g., absolute orientation) of the animal, for example, in the NESW plane.

As described above, the mechanism 200 may include a GPS, proximity sensor, location system, etc. The GPS may track a position of the animal. For example, the GPS may indicate that the animal is inside a home, outside a home, etc. For example, the GPS may indicate that the animal is within a park (e.g., a dog park), within an exercise area (such as an exercise area of a dog boarding kennel), within a crated area, etc. The movement of the animal may be associated with the location of the animal. For example, the caloric requirements of an animal may be increased if the animal is running outside of the home.

Mechanism 200 may send data relating to an animal to a server, electronic device (e.g., mobile phone of pet parent or caregiver), and the like. For example, mechanism 200 may send motion data, orientation data, location data, etc., to a server, electronic device, etc. The server may perform computations of the data, for example, to determine the present and/or future caloric requirements of the animal based on factors described herein, such as the present and/or past activity levels of the animal or movements of the animal. The server may store previous caloric requirements determined for the animal, predict present and/or future caloric requirements for the animal (assuming the animal will have the same or similar activity level and/or movements as the animal had in the past).

The determined caloric requirements for the animal may be compared with other animals that may be similarly situated (e.g., same breed, similar activity level, etc.). The server may determine how accurate the determined caloric requirements were for the other animals, for example, based on how effective the determined caloric requirements were for correcting health disorders (such as obesity). Based on how accurate the determined caloric requirements were for the other animals, the server may perform adjustments for the determination of the animal. The server may be configured to communicate the data to the user and/or to one or more other parties (e.g., a veterinarian, pet parents, care givers, etc.). In examples, an electronic device (e.g., the care giver's mobile phone) may perform computations of the data to determine caloric requirements of the animal. The electronic device may be configured to communicate the data to the user and/or one or more other parties (e.g., a veterinarian, spouse, etc.).

The mechanism 200 may have a biometric monitoring sensor. The biometric monitoring sensor may be configured to determine body measurements and/or calculations of the animal. For example, temperature sensor and/or heart rate sensor may be used to determine the body temperature of the animal and/or the heart rate of the animal. The biometric monitoring sensor may include a sensor to determine the respiration, glucose level, and/or other biometric condition of the animal. The biometric monitoring sensor may be located on the activity collar or on another device position on or about the animal. The biometric data may be used to determine caloric requirements of the animal.

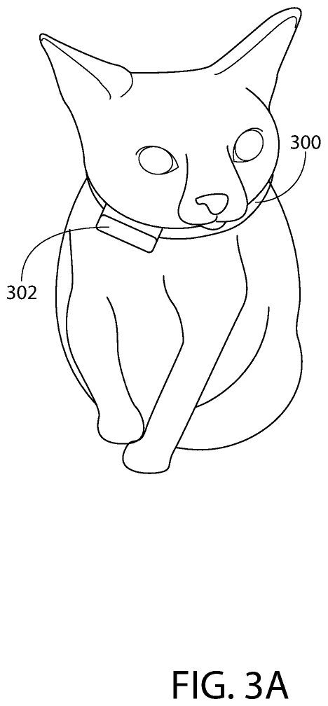

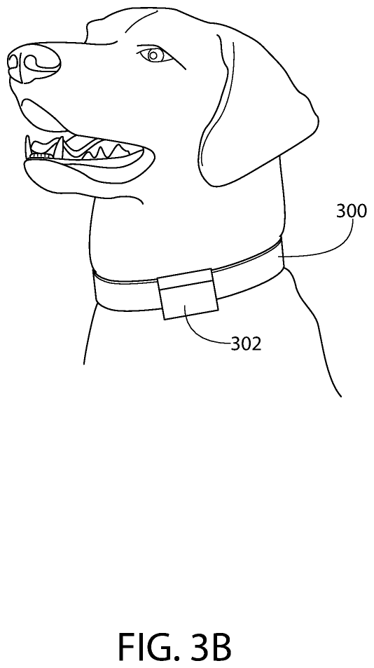

FIGS. 3A, 3B show examples of mechanism 300. As shown on FIG. 3A, a cat may wear the mechanism 300 in the form factor of an activity collar, in other examples other animals can wear mechanism 300, such as the dog shown wearing the activity collar in FIG. 3B. Although the example mechanism 300 as shown on FIGS. 3A/3B in the form factor of a collar, it should be understood that the collar is for illustration purposes only and mechanism 300 may be any device (e.g., wearable device) that may come in other form factors besides a collar, as described herein. For example, mechanism 300 may be a harness, jacket, vest, hat, gloves, contact lenses, rings (e.g., earrings), chip (e.g., electronic chip), or any other device (or combinations of devices) that can be worn on the outside (or inside) of an animal.

As described herein, the mechanism 300 (e.g., activity collar) may have one or more sensors 302, such as an accelerometer. The sensor 302 may be coupled to the mechanism 300, for example, on an outside of the mechanism 300. In other examples, the sensor (e.g., accelerometer) may be integrally formed within the mechanism 300. Sensor 302 may include a location sensor. The location sensor may be located on the animal (e.g., worn by the animal) or positioned upon a surface that is not the animal. The location sensor may determine the absolute location of the animal or a relative location of the animal.

The sensors and other devices may be used to determine the activity level and/or movement of the animal, such as the direction, speed, acceleration, duration, etc. in which the animal is moving. The activity level and/or movement of the animal may be determined based on motion data, orientation data, location data, etc., of the animal. The sensors and other devices may be used to determine the location at which an animal is moving.

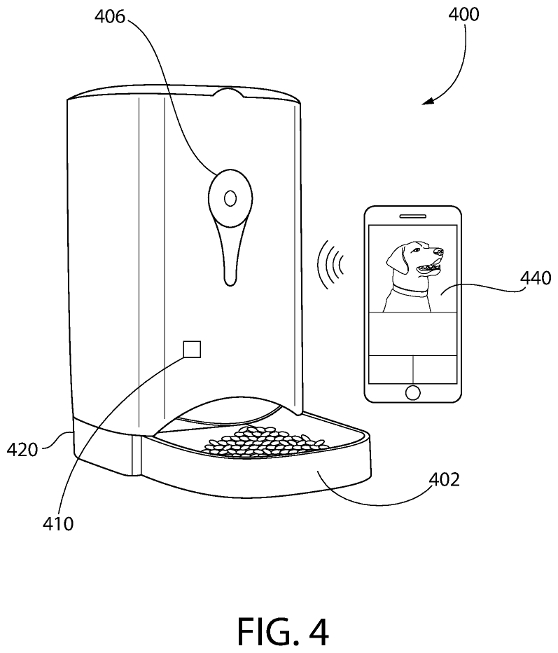

FIG. 4 shows an example feeding system 400 that may be used to monitor an animal feeding behavior and/or habits to determine caloric requirements of the animal. The animal's feeding behavior and/or habits may be monitored via one or more of the sensors, devices (e.g., measuring devices), etc., described herein. For example, feeding bowl 402 may include a device 410, which may be communication circuitry. Communication circuitry may be Bluetooth, RFID, Wi-Fi and other wireless technologies. The feeding bowl 402 may communicate with an activity collar (such as activity collar 300) and/or a server.

Feeding bowl 402 may communicate with one or more devices, such as an activity collar of the animal. Based on the communication between feeding bowl 402 and the activity collar, feeding bowl 402 may recognize the animal and/or identify the profile of the animal. Feeding bowl 402 may recognition the animal and/or identify the profile of the animal as the animal approaches feeding bowl 402, remains near feeding bowl 402, and/or leaves feeding bowl 402. Feeding bowl 402 may distinguish one or more animals, for example, based on profiles of activity collars situated on animals. Based on distinguishing the animals, feeding bowl may ensure that the recommended type of food, amount of food, etc. is provided to the animal. In examples, feeding bowl 402 may ensure that a type of food accessible by one animal is not accessible by one or more animals. For example, feeding bowl may place a barrier (such as a door) between food that is not intended for an animal, and remove the barrier when the intended animal is near the food.

Feeding bowl 402 may communicate directly with a portable electronic device of the user (such as mobile phone 440), or such communication may occur indirectly via a server and an application, such as a web application. Feeding system 400 may include one or more devices 410, which may include a proximate sensor, a scale, a microphone etc., to track the eating behavior and/or eating habits of the animal over time. For example, a proximate sensor may be used to determine the location (e.g., relative location of the animal with respective to one or more locations, such as feeding area). A scale may determine the weight of the animal, which may include the weight before a feeding, after a feeding, during a feeding, and the like.

A microphone may be used to determine sounds around the feeding area, such as sounds to alert the feeding system 400 that the animal is entering the feeding area, leaving the feeding area, present within the feeding area, and the like. Microphone may be used to receive sounds of the animal eating, such as the speed (e.g., fast, slow) in which the animal is eating, the breathing of the animal while feeding, the crunchiness or softness of the food in which the animal is eating, noises that may distract the animal while feeding, and the like. Feeding system 400 may include a camera 406 to take photographs of the animal eating. The photographs may determine facial expressions of the animal while feeding, such as if the animal is very hungry while eating (e.g., beginning eating, finishing eating), in pain while eating, eating with one or more other animals (which may impact the amount of food eaten), in a rush to eat, appearing to be ill while eating, and the like.

The feeding bowl 402 may include a delay to minimize incidences of false approaches (e.g., where an animal is merely walking by the feeding bowl 402, as opposed to approaching the feeding bowl 402 to eat). The feeding bowl 402 may include a memory, controller, and local user interface/display. The animal's behaviors and/or habits relating to the animal's feeding may also, or alternatively, be monitored at the feeding area using the sensors, devices (e.g., measuring devices), etc., located on an animal (e.g., an activity collar), as described herein. Although the feeding area is shown as a feeding bowl 402, it should be understood that a feeding bowl may be different form factors than shown on FIG. 4, and may include one or more other types of bowls, such as a drinking bottle or drinking bowl.

As shown on FIG. 4, food bowl 402 may be an area designated for an animal (e.g., a cat or dog) to eat and/or drink. As described herein, the food bowl 402 may have one or more devices, which may be sensors. The one or more sensors may be located on, above, under etc., the food bowl 402. The one or more sensors may be located on a portion of the feeding system 400 and/or may not be located on a portion of the feeding system 400. Device 410 may be one or more motion sensors (such as an accelerometer, a gyroscope, a magnetometer, etc.), proximity sensors, orientation sensors, location sensors, and/or one or more other sensors, as described herein.

Feeding system 400 may have a measuring device, such as measuring devices 420. Measuring device 420 may be a scale that may determine the amount of food consumed by a pet, for example, by determining the weight and/or volume of the food stored by the feeding system 400 and the weight and/or volume of the food removed by the animal via one or more feedings. As described herein, measuring devices 420 may be one or more weighing scales, weight transducers, force transducers, displacement transducers, pressure transducers, weight sensors, force sensors, displacement sensors, pressure sensors, real time clocks, timers, counters, and/or a combination thereof. Measuring device 420 may be used to determine the weight and/or volume of the food consumed by an animal. For example, measuring device 420 may determine the weight and/or volume of food stored in the feeding system 400 and the weight and/or volume of the food removed from the feeding system, for example, via feeding bowl 402.

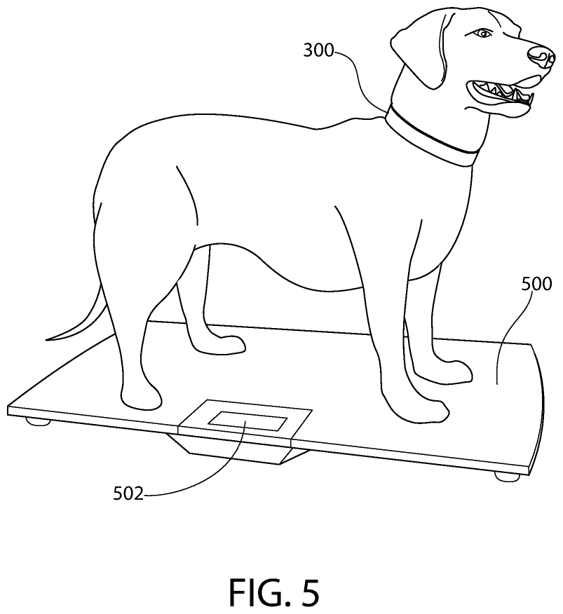

FIG. 5 shows an example weighing device 500 that may be used to monitor and/or measure the weight and/or pressure of the animal. Weighing device 500 may be used to measure the weight of the animal. The weight of the animal may be provided to a display, such as display 502, and/or the weight of the animal may be provided to a remote device, such as a user device, server, etc. So that an accurate weight reading may be performed, weighing device 500 may be placed at a location in which the animal may stay still. For example, weighing device 500 may be placed at (e.g., under) a bed of the animal, a food bowl (such as food bowl 402) of the animal, etc. The weight of the animal may be identified, determined, and/or stored based on an occurrence (e.g., every time the animal does not move on the scale), on a periodic (e.g., every twenty four hours), at a time of day (e.g., in the morning), and the like. The weight may be associated with an animal (e.g., a particular animal) based on the profile of the animal stored on a collar (e.g., activity collar 300) worn by the animal.

A health condition of the animal may be determined and/or managed based on the weight of the animal. For example, if the animal is within a defined weight for the animal's breed, body type, condition, morphometric measurements (such as leg length, head circumference), and/or activity level, the animal may be determined to be healthy. If the weight of the animal is outside of a defined recommended weight (or trending towards the outside of the defined recommended weight) for the animal's body type, breed, condition, and/or activity level, the animal may be determined to be unhealthy (e.g., obese, overweight, or underweight). The caloric requirement of the animal may be based on the weight of the animal and/or the weight in which the animal is trending. For example, an animal that is within a healthy weight range may be recommended a number of calories presently consumed by the animal. If an animal is outside a healthy weight range or trending outside a healthy weight range of the animal, an adjustment of calories may be required for the animal. For example, an obese animal may be required to consume less calories and an extremely lean animal may be required to consume more calories. As another example, an animal may be required to consume less calories if the weight of the animal is trending towards obesity, and the animal may be required to consume more calories if the weight of the animal is trending towards too small of a weight. By addressing the calorie intake of the animal before the weight of the animal is outside of a healthy weight, the health of the animal can be managed before the animal's health is negatively impacted by an unhealthy weight.

Mathematical and/or algorithmic techniques, such as bivariate, multivariate and trend analysis, may be used to formulate a trend of the animal events (e.g., activity level and/or movement of the animal). Data collected over time and processed can represent a typical profile of behavior of an animal. The behavior and habits of the animal may be used to determine the animal's caloric requirements. For example, an animal that frequently exhibits a high activity level may require more calories than an animal that exhibits a burst of high activity after a period of low activity. Also, or alternatively, trend analysis may be used to determine whether the monitored behavior, habits, etc. of the animal are random, or whether a trend may be developing.

Notifications may be delivered to portable electronic device, for example, in the form of an electronic mail message sent to a user-specified electronic mail address, a text message sent via SMS (Short Message Service) to a user-specified mobile phone number, a calendar reminder set up by the system in a user-specified calendar, phone calls to a user-specified mobile or landline phone number, messages by a mobile phone application of a user's mobile phone, etc.

The time and/or duration of an animal's movements may be recorded. For example, a spike (or reduction) in the amount of activity level of an animal may be recorded and sent to the pet owner so that the pet owner may ensure that the pet consumes (e.g., timely consumes) an amount of calories commensurate with the animals activity level. Records relating to the animal may be stored and/or may be presented, for example, via a textual or graphical format.

A profile of the animal may be accessed via a portable electronic device. The portable electronic device may provide a user interface, for example, via an application downloaded on the portable electronic device. A user may create a profile associated with the animal. The application may display the animal's profile and/or may be facilitate the uploading of monitoring information of the animal. Icons or symbols displayed on the application may designate an activity level, movement, or caloric requirement of an animal that is being monitored and/or tracked. Such data may be displayed in graph form for ease of reference.

A calorie recommendation of the pet may be provided based on one or more parameters described herein. For example, a caloric recommendation for a particular pet may be provided based on an activity of the pet, a weight of the pet, a food consumption (e.g., food consumption amount, food consumption behavior, food consumption habit) of the pet, etc., as described herein. The calorie recommendation may include a type of food recommendation, a brand of food recommendation, an amount of food recommendation (e.g., an amount of food recommended to be consumed per day, twice per day, per week, etc.), contents of a food recommendation (e.g., a pet food with chicken, a pet food without dairy), etc.

In examples in which the calorie recommendation includes a type and/or brand of the food recommended for the animal, the system may order (e.g., automatically) order the food prior to the pet owner being out of the pet food. For example, the system may identify the number of calories required for an animal for a week, a month, etc. If the system knows how many calories are stored at the pet parent's home (e.g., via weighing the amount of food provided in a feeding system, identifying a history of the amount of food requested in previous orders), the system may be able to determine when the food is expected to be exhausted. The system may order pet food based on one or more factors, such as how much food is available to the pet, how much food the pet is expected to consume, shipping times, and the like. The system may automatically order pet food in examples, although in other examples the system may provide an indication to the owner when the pet food is expected to be exhausted, so that the pet owner may order additional food.

Calorie recommendations and/or requirements, as described herein, may be based on one or more parameters related to the pet. For example, a high amount of a calories may be recommended for a pet that is very active and is below a weight threshold, a low amount of a calories may be recommended for a pet that is somewhat active and is above a weight threshold. Thresholds (e.g., weight thresholds) may be based on databases defining the desired weights of animals based on the animal's body type, breed, height, length, age, activity level, etc. In another example a low amount of a calories may be recommended for a pet that is obese and that is not very active. The low amount of calories may result in the animal being placed on a diet (e.g., reduced diet) based on more than parameter relating to the animal, such as the activity level of the animal and the weight of the animal.

As described herein, the caloric recommendation of the animal may include a type of food recommendation, such as a food intended for active animals, animals with predefined disorders (e.g., kidney disorders, ligament disorders), animals who are dieting, etc. Caloric recommendations may include a brand of a food recommendation, which may include brands in which the animal's veterinarian is comfortable with, value brands or premium brands, brands that are on sale, brands in which the veterinarian receives a portion of the sale, etc. Caloric recommendations may be based on information relating to the animal's medical health and/or medical history, for example, as stored in a profile of the animal. Caloric recommendations may include an amount of food recommendation (e.g., an amount of food recommended to be consumed per day, twice per day, per week, etc.), which may include a weight of the food to be recommended (e.g., 1 pound) per day, a volume of liquid to be recommended, a volume of food (e.g., the amount of food that may fill a container of food system 500), etc. Caloric recommendations may include contents of the food to be recommended. Such contents may include ingredients, such as food having chicken and/or rice, a pet food not containing beef, etc.

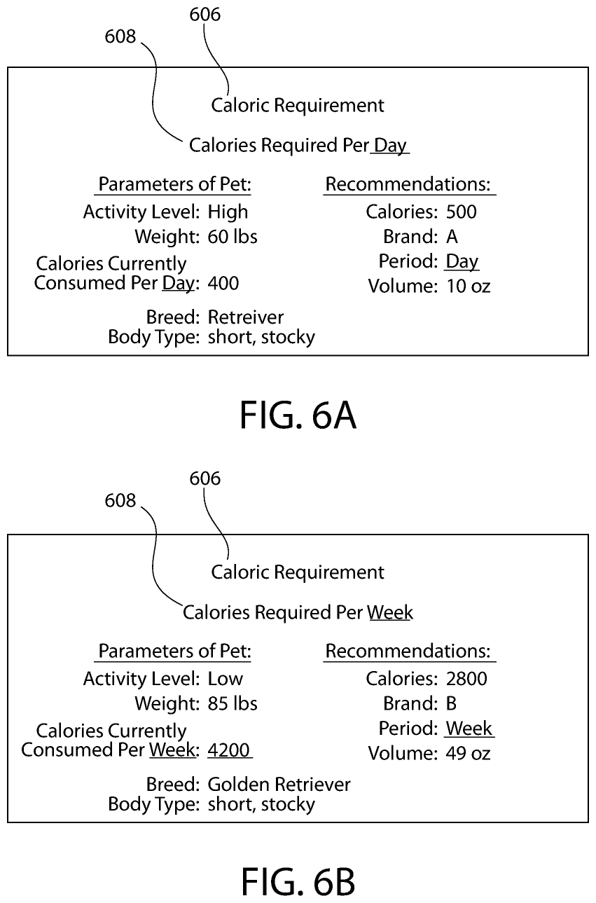

FIGS. 6A-6B show example screenshots of a system providing caloric (e.g., calorie) recommendations of an animal. Caloric requirements may include the amount of calories recommended, the type of food (e.g., dry, wet, chicken, etc.) to provide the recommended calories, the brand of food to provide the recommended calories, duration of feeding, etc. The screenshots may be provided on a portable electronic device, for example. The information used to determine the caloric requirements and/or the caloric requirements, may be stored on an external device, such as a server. The screenshots may provide indications of one or more parameters related to the animal, such as the body type, breed, age, weight, growth, activity level, calories presently and/or previously consumed, movement (e.g., steps), behavior (e.g., anxiety, aggression), sleep schedules (e.g., lack of sleep, an overabundance of sleep, sleep disturbances), and conditions (e.g., pregnancy condition, dementia, and/or other health condition) of the animal. The parameters related to the animal may be provided via one or more sensors, weighing scales, feeder systems, and/or user inputs. In examples the parameters related to the animal may be provided via one or more external sources, such as via user inputs, external servers, and the like. The parameters and caloric requirements shown on the screenshots are for illustration purposes only and are not limiting. In examples, other parameters and/or other requirements (e.g., water requirements) may be provided to the user.

FIG. 6A shows an example screen shot of data collected by one or more elements (such as mechanism 200, feeder system 400, and/or weighing scale 500) worn by an animal or proximate to the animal for determining a caloric requirement of the animal. Category 606 shows the Caloric Requirement is being determined for an animal. In other examples, category 606 may show other categories, including the identity (e.g., name) of the animal, the amount of activity recommend for the animal, the amount of liquid that an animal should ingest, etc. The screenshots may be provided on a display, such as on a display of a portable electronic device.

As shown on FIGS. 6A, 6B, a time duration (e.g., period) 608 may be provided. Time period 608 may define the period of the data provided. For example, the period of the data may include the duration in which the animal's activity level, movements, weights, calories consumed, have been monitored and/or determined. The period of data may include the recommended calories to be provided to the animal. For example, the period of data may be of the calories that should be provided to an animal per day.

Using the example shown on FIG. 6A, the body type, breed, weight, activity level, and/or caloric requirement is provided for the time period of a day. The caloric requirement may be the required caloric intake for a day, week, or any other period. The screenshots may include parameters of the pet and/or recommendations (e.g., recommendations based on the parameters of the pet). For example, as shown on FIG. 6A, the parameters of the pet may include the activity level of the pet, the weight of the pet, the calories currently (or presently) consumed by the pet, the body type of the pet, and/or the breed of the pet. Based on the parameters, the recommendations may include the calories required of the pet, the brand of pet food in which the calories is recommended to be provided, the period in which the calories should be distributed, and/or the volume of the pet food (e.g., if the recommended pet food is provided). Of course, the parameters of the pet and/or the recommendations (including the numbers provided), is for illustration purposes only. For example, the parameter activity level of the animal may be based on a different scale (e.g., may be based on a number scale), may be more or less precise, etc. Different (including more or less) categories of data, time periods, etc., may be displayed.

Using the example shown on FIG. 6B, category 606 may relate to caloric requirements for a time period 608 of a week. The weekly caloric requirements may be based on a time period, for example, from Jul. 15, 2020 to Jul. 22, 2020. As described herein, in other examples time period may be any time period, including a single day (e.g., Jul. 15, 2020), a few days, a week, etc. One or more of the recommendations may be modified based on the time period 608, such as the recommended number of calories.

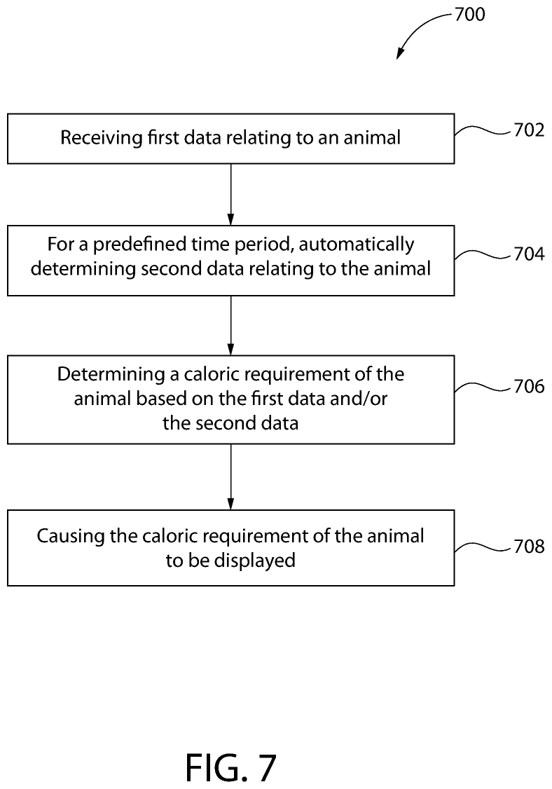

FIG. 7 describes an example method 700 of determining an amount of calories required of an animal, as described herein. The amount of calories required by the animal may be based on one or more parameters relating to the animal, such as the activity level of the animal, the movement of the animal, the weight of the animal, the amount of calories consumed (e.g., presently consumed or previously consumed) by the animal, the body type of the animal, the breed of the animal, the age of the animal, the weight of the animal, the growth of the animal (e.g., growth of the animal within a predefined time, such as within the past month or year), a medical disorder and/or illness of the animal (e.g., a kidney disorder, a ligament and/or joint disorder, etc.), behavior of the animal (e.g., anxiety, aggression), a sleep schedule of the animal (e.g., lack of sleep, an overabundance of sleep, sleep disturbances), a condition (e.g., lactation or pregnancy condition) of the animal, etc. One or more of the parameters relating to the animal may be automatically received and/or determined. For example, the activity level of the animal, the movement of the animal, the weight of the animal, the amount of calories consumed (e.g., presently consumed or previously consumed) by the animal, and the like, may be automatically determined and/or received by a sensor or other electronic device. In other examples the data, such as the age, medical disorder, name, etc., of the animal may be provided via a user (e.g., via a mobile device).

At 702, first data relating to an animal may be received. For example, first data relating to the body type of the animal, the breed of the animal, a condition (e.g., a medical condition, such as a pregnancy condition, lactating condition, or medical disorder) of the animal, an identity of the animal, an age of the animal, a weight of the animal, etc., may be received. The first data relating to the animal may determine the number of calories that is required by the animal. For example, an animal having a stocky body type may require more calories than an animal having a thin body type, an animal belonging to a large breed may require more calories than an animal belonging to a smaller breed, an animal having a high energy (e.g., belonging to a breed of a high energy animal) may require more calories than a more relaxed animal, a pregnant animal may require more calories than a non-pregnant animal, an animal with a kidney disorder may require less calories (and/or more water) than an otherwise healthy animal, etc. The first data may be received by and/or provided to a server and/or a user device (e.g., a user device of a pet owner or a veterinarian).

At 704, second data relating to an animal may be determined and/or received. The second data may relate to a predefined time period, such as one or more hours, days, weeks, months, years, and the like. The second data may relate to a time of day, such as a morning, afternoon, evening. The second data may be automatically determined and/or received, for example, via one or more elements. Elements may include one or more sensors, measurement devices, and/or storage devices, as described herein. For example, elements may include one or more accelerometers, gyroscopes, magnetometers, weighing scales, weight transducers, force transducers, displacement transducers, pressure transducers, weight sensors, force sensors, displacement sensors, pressure sensors, load cells, photographic cameras, video cameras, camcorders, contact thermometers, non-contact thermometers, and a combination thereof.

Second data may relate to calories consumed. In examples, second data relating to calories consumed (e.g., previously and/or currently consumed) may be determined and/or received (e.g., automatically determined and/or received) via a feeding system, such as feeding system 400. In addition, or alternatively, second data relating to the animal may be automatically determined and/or received via optical sensors, optical reflecting sensors, LED/photodiode pair optical sensors, LED/phototransistor pair optical sensors, laser diode/photodiode pair optical sensors, laser diode/phototransistor pair optical sensors, optocouplers, optical fiber coupled optical sensors, magnetic sensors, weight sensors, force sensors, displacement sensors, pressure sensors, various proximity sensors, such as inductive proximity sensors, magnetic proximity sensors, capacitive proximity sensors, and/or a combination thereof. Second data relating to an animal may be received and/or determined circuitry, such as communication circuity including Bluetooth, RFID, Wi-Fi and other wireless technologies.

Data (e.g., first data, second data) relating to the animal may include the activity level of the animal, the movement of the animal, the weight of the animal, the amount of calories consumed (e.g., presently consumed or previously consumed) by the animal, the breed of the animal, the body type of the animal, a condition (e.g., lactation, pregnancy, or other medical condition) of the animal, etc. For example, the activity level of the animal may be determined (e.g., automatically determined) via an accelerometer placed on the animal, such as a collar of the animal. The accelerometer may determine the steps, distance, speed, direction, etc., traveled by the animal in a predefined period, such as in a day, week, etc. Based on the steps, distance, speed, direction, etc., traveled by the animal, the activity level of the animal may be determined. For example, an animal traveling a distance greater than a predetermined distance may be determined to have a high activity status, while an animal traveling a distance less than a predetermined distance may be determined to have a low activity status. Based on the activity level of the animal and other parameters of the animal (such as the breed, age, size, weight, calories consumed), a recommended (e.g., required) amount of calories for the animal may be provided.

A feeding system, such as feeding system 400, may be used to determine (e.g., automatically determine) the amount of calories consumed by the animal at the present time and/or a previous time. The feeding system may determine the amount of calories consumed based on the weight of the food consumed by the animal (via a weighing of the food removed from the feeding system by the animal), the volume of the food consumed by the animal, the freshness of the food consumed by the animal, etc. Based on the amount of calories consumed by the animal (as well as additional parameters, such as breed, body type, activity level, and weight of the animal), a recommended (e.g., required) amount of calories for the pet may be provided.

A weighing scale, such as weighing scale 500, may be used to determine (e.g., automatically determine) the weight of the animal. The weight of the animal may be compared against suggested weights for a breed of the animal, a body type of the animal, a size (e.g., length) of the animal, an age of the animal, etc. Based on the weight of the animal and other parameters of the animal (such as the breed, body type, age, size, activity level, calories consumed), a recommended (e.g., required) amount of calories for the pet may be provided. The parameters relating to the animal, such as the activity level, weight, calories consumed, etc., may be associated with a duration, such as the daily activity level of the animal, the daily weight of the animal, the daily calories consumed by the animal, etc. The calories recommend for the animal may relate to the same duration as the parameters relating the animal. For example, if the daily activity level is provided for the animal, the daily recommended calories may be provided. In other examples, the calories recommend for the animal may have a different duration than the parameters relating the animal.

At 706, the calories recommended and/or required for the animal (e.g., recommended for the animal for a predetermined time period) may be determined. The calories recommended and/or required for the animal may be based on the first data (e.g., data relating to the body type of the animal and/or breed of the animal), as described herein. The calories recommended and/or required for the animal may also, or alternatively, be based on the second data relating to the animal, such as the activity level of the animal, weight of the animal, calories consumed by the animal, conditions of the animal, etc. For example, the distance traveled by the animal along with one or more parameters (such as the body type and/or weight of the animal) may be converted to the calories exerted by the animal. By determining the calories exerted by the animal, it may be determined the amount of calories that the animal requires (e.g., to replace the calories lost by the animal). For example, the calories required for a defined future time period may be based on the amount of calories exerted by the animal during a previous time period. The previous time period and the future time period may be the same amount of time or a different amount of time.

The daily calorie expenditure of the animal may be extrapolated for one or more days, weeks, months, etc. The amount of calories exerted during a time period may be determined as the amount of calories required for a present and/or future time period (e.g., same time period). For example, if 400 calories were exerted by the animal during the past twenty-four hours, it may be assumed that 400 calories may be required in the next twenty-four hours. In another example, if the animal's activity results in an additional 400 kcals/day being required by the animal (e.g., required on average over a period of time), it may be determined that 400 kcals/day may be required (e.g., on average) to maintain the weight of the animal over the subsequent period of time until the next calculation is made. Such a determination may be required as activity is variable and the need to account for recent activity and/or the need to revise calculations may be based on recent data. For example, if 2,500 calories were exerted by the animal during the past seven days, it may be assumed that 2,500 calories may be required in the next seven days.

At 708, the caloric requirement of the animal may be caused to be provided (e.g., displayed). For example, the caloric requirement may be displayed on a display of a portable electronic device, such as a mobile phone, a tablet, or a mobile phone. The caloric requirement may be sent via one or more electronic methods, such as via SMS messages, email, and the like. The caloric requirement may relate to a daily caloric requirement of the animal, a weekly requirement of the animal, and the like. Subsets of the caloric requirement of the animal may be provided. For example, the daily caloric requirement may be divided into a caloric requirement of the animal for the morning and a caloric requirement for the evening. The user may use such subset data to provide the food required for the animal in the morning and in the evening.

The caloric requirements of the animal may be provided in one or more form factors. For example, the amount of recommended calories may be provided (e.g., provided for a predefined duration). The brand of pet food in which the recommended amount of calories may (e.g., may also) be provided. For example, 500 calories of brand ‘A’ pet food may be recommended and/or required. A volume of the recommended pet food (e.g., brand of pet food) may be provided. For example, twelve ounces of brand ‘A’ pet food may relate to 400 calories. In such example, if 400 calories is required (e.g., recommended) for the animal, twelve ounces of brand A pet food may be recommended and/or required for the animal.

The caloric requirements of the animal may be provided to a veterinarian and/or a pet parent via one or more methods. For example, notifications of the caloric requirement may be delivered to portable electronic device, for example, in the form of an electronic mail message sent to a user-specified electronic mail address, a text message sent via SMS (Short Message Service) to a user-specified mobile phone number, a calendar reminder set up by the system in a user-specified calendar, phone calls to a user-specified mobile or landline phone number, messages by a mobile phone application of a user's mobile phone, etc. Upon receiving the caloric requirements, the receiver may provide the required number of calories to the animal. The results of providing the required calories may be received and/or stored for a determination of the success of the animal being fed the required number of calories. Depending on how successful the required number of calories is in reducing health disorders (e.g., obesity), the amount of calories required for the animal may be reduced, increased, or may remain the same.

While the invention has been described with respect to specific examples including presently preferred modes of carrying out the invention, those skilled in the art will appreciate that there are numerous variations and permutations of the above described systems and techniques. It is to be understood that other embodiments may be utilized and structural and functional modifications may be made without departing from the scope of the present invention. Thus, the spirit and scope of the invention should be construed broadly as set forth in the appended claims.

1. A method comprising:
receiving, via one or more processors, animal characteristic data comprising a breed of an animal or a condition of the animal;
receiving, via one or more elements, durational parameters of the animal for a first predetermined time duration, wherein the durational parameters comprise at least one of an activity level of the animal during the first predetermined time period, a weight of the animal during the first predetermined time period, or an amount of calories consumed by the animal during the first predetermined time period;
determining, via one or more processors, a caloric requirement of the animal during a second predetermined time period, the caloric requirement of the animal being based on the animal characteristic data of the animal and the durational parameters of the animal during the first predetermined time period; and
causing the caloric requirement of the animal during the second predetermined time period to be displayed via a display device.

2. The method of claim 1 wherein the elements comprise at least one of an accelerometer, a gyroscope, a magnetometer, or a global positioning system (GPS) device, a feeding system, or a weighing scale.

3. The method of claim 2 wherein the accelerometer is coupled to a collar attached to the animal.

4. The method of claim 1 wherein the first predetermined time period and the second predetermined time period have a same duration.

5. The method of claim 4 wherein the same duration is a duration of twenty-four hours.

6. The method of claim 1 wherein the first predetermined time period and the second predetermined time period have a different duration.

7. The method of claim 6 wherein the first predetermined time period has a first duration and the second predetermined time period has a second duration, the first duration being longer than the second duration.

8. The method of claim 6 wherein the first predetermined time period has a first duration and the second predetermined time period has a second duration, the first duration being shorter than the second duration.

9. The method of claim 1 wherein the condition of the animal comprises at least one of a pregnancy condition of the animal or a lactating condition of the animal.

10. The method of claim 1 wherein the animal condition comprises a medical disorder of the animal.

11. The method of claim 1 further comprising determining a trend of caloric requirements of the animal based on the determined caloric requirement of the animal and the amount of calories consumed by the animal during the first predetermined time period.

12. (canceled)

13. A system for determining calories required of an animal comprising:
one or more elements configured to receive durational parameters of the animal for a first predetermined time duration, wherein the durational parameters comprise at least one of an activity level of the animal during the first predetermined time period, a weight of the animal during the first predetermined time period, or an amount of calories consumed by the animal during the first predetermined time period; and
one or more processors configured to:
receive animal characteristic data comprising a breed of the animal or a condition of the animal;
determine a caloric requirement of the animal during a second predetermined time period, the caloric requirement of the animal being based on the animal characteristic data of the animal and the durational parameters of the animal during the first predetermined time period; and
cause the caloric requirement of the animal during the second predetermined time period to be displayed via a display device.

14. The system of claim 13 wherein the elements comprise at least one of an accelerometer, a gyroscope, a magnetometer, or a global positioning system (GPS) device, a feeding system, or a weighing scale.

15. The system of claim 14 wherein the accelerometer is coupled to a collar attached to the animal.

16. The system of claim 13 wherein the first predetermined time period and the second predetermined time period have a same duration.

17. (canceled)

18. The system of claim 13 wherein the first predetermined time period and the second predetermined time period have a different duration.

19. The system of claim 18 wherein the first predetermined time period has a first duration and the second predetermined time period has a second duration, the first duration being longer than the second duration.

20. The system of claim 18 wherein the first predetermined time period has a first duration and the second predetermined time period has a second duration, the first duration being shorter than the second duration.

21. (canceled)

22. (canceled)

23. The system of claim 13 further comprising determining a trend of caloric requirements of the animal based on the determined caloric requirement of the animal and the amount of calories consumed by the animal during the first predetermined time period.

24. The system of claim 13 wherein at least one of the one or more processors is located at a server.
